(19) United States
(12) Patent Application Publication
(10) Pub. No.: US 2012/0129792 A1
(43) Pub. Date: May 24, 2012

(54) AGENT FOR ELIMINATING MULTIDRUG RESISTANCE

(76) Inventor: Vasiliy Vyacheslavovich LEBEDEV, Moscow (RU)

(21) Appl. No.: 13/115,472

(22) Filed: May 25, 2011

Related U.S. Application Data
(63) Continuation of application No. PCT/RU2009/000639, filed on Nov. 23, 2009.

(30) Foreign Application Priority Data
Nov. 25, 2008 (RU) 2008146409

(51) Int. Cl. A61K 38/08; A61P 31/00; A61P 35/00

(52) U.S. Cl. 514/21.8

(57) ABSTRACT

The invention belongs to medicine, namely to pharmacology, and may be used to overcome multiple drug resistance in oncological and infectious patients undergoing long-term chemotherapy. Essence of the invention: proposed herein as a therapeutic agent intended to overcome multiple drug resistance is a hexapeptide with the following structural formula: lysyl-histidyl-glycyl-lysyl-histidyl-glycine.

### CROSS-REFERENCE TO RELATED APPLICATIONS

This Application is a Continuation application of International Application PCT/RU2009/000639 filed on Nov. 23, 2009, which in turn claims priority to Russian patent application RU2008146409 filed on Nov. 25, 2008 entitled, both of which are incorporated herein by reference in their entirety.

### FIELD OF THE INVENTION

The invention belongs to medicine, namely to pharmacology, and may be used to overcome multiple drug resistance in oncological and infectious patients undergoing long-term chemotherapy.

### BACKGROUND OF THE INVENTION

Multiple drug resistance (hereinafter referred to as MDR) of tumour cells, gram-negative bacteria, and the protozoa to cytostatic and antimicrobial agents is known to develop resulting from the natural cellular defence mechanisms. Such mechanisms through ATP-dependent transport proteins ensure intensive expulsion of all noxious compounds out of cells, including chemotherapeutical agents Enhanced intensity of the function of the cellular transport pump during chemotherapy brings about a sharp decrease in the concentration of chemical agents in the cell, thus dramatically diminishing therapeutic action of drugs in patients suffering from bacterial, parasitic and fungal infections, as well as oncological diseases. The above-mentioned broad spectrum of diseases whose management is complicated and sometimes turns out even impossible owing to the development of MDR is clearly indicative of an urgent need to devise novel efficient agents intended to inhibit or circumvent MDR.

In the mammalian cells, responsible for MDR are primarily two types of transport proteins: multidrug resistance proteins (MRPs) and P-glycoprotein (P-gp). Both MRPs and P-gp are found in various-origin tumours: lymphomas, sarcomas, carcinomas, and neuroblastomas.

The substrates for the both types of transport proteins appear to be a broad spectrum of antineoplastic drugs: anthracycline, etoposide, vincristine, taxol, etc. However, unlike P-gp, the MRP transports various substances from cells in the form of conjugates with glutathione or glucuronic acid. Resulting from the function of the transport proteins, cytostatic agents enter the extracellular space, and tumour cells acquire resistance simultaneously to a variety of drugs.

A wide variety of substances is known to suppress the activity of the transport to protein P-gp and inhibit excretion of chemical agents from cells. By their physicochemical properties, practically all MDR-inhibiting compounds we know of turn out to share common traits, including but not limited to low molecular weight, intrinsic hydrophobic physicochemical features, and the presence of the aromatic ring in the molecule. To MDR inhibitors belong therapeutic agents of the following groups: calcium channel blockers, calmoduline inhibitors, coronary vasodilators, indole alkaloids, hormones, cyclosporines, surfactants, and antibiotics [Krishna R., Mayer R. D., Eur. J. Pharmacol. Sci. 2000. Vol. 11. No 4. 265-283].

However, the substances referred to above appear to display a low specific activity in relation to inhibiting MDR of tumour cells. Thus, calcium channels blockers, for instance verapamil, are known to exhibit an in vitro inhibiting activity at a dose of approximately 10 μM.

Another known group of agents—calmoduline antagonists: trifluorperasine, chlorpromazine, prochlorperazine appear to show the capability of decreasing resistance of tumour cells to chemotherapeutic agents only at relatively high concentrations amounting to about 5-50 μM. Although MDR inhibitors have been identified, none of them has hitherto been proven clinically useful without adverse side effects. Administration of the known drugs at the acting concentrations to laboratory animals requires such high doses that result in either lethal outcomes or severe complications [Sandor V., Fojo T., Bates S. E., Drug Res. Updates 1998. V. 1, 190-200]. Severe toxic reactions of the known MDR inhibitors do not allow of their use in patients to overcome MDR. Therefore, up to now there are no drugs providing possibility to reliably circumvent MDR in oncological or infectious patients.

The development of MDR in tumour cells is mediated not only by transporters known as P-pg but also by other transport agents, including multidrug resistance proteins (MRPs). The latter confers resistance to the same chemical compounds, as does P-gp, but with somewhat another spectrum of cross-resistance. Multidrug resistance-associated proteins belonging to the MRP family, unlike the P-gp proteins, have a wide interspecific representation and appear to control the development of MDR of tumour cells as well as in helminths, protozoa, and bacteria [Borst T., Kool M., Evers R. Sem. Cancer Biol. 1997. V. 8, 205-213]. Therefore, therapeutic agents capable of influencing the activity of transport proteins MRPs have a broader spectrum of action as compared with the drugs influencing the activity of the transport proteins P-gp. The former may influence not only the formation of MDR of tumour cells but also the manifestation of multiple drug resistance in helminths, protozoa, including malaria plasmodium, and bacteria.

Known as a therapeutic agent is a cyclohexapeptide with the following structural formula: cyclo-lysyl-histidyl-glycyl-lysyl-histidyl-glycine [Russian Federation Patent RU 2157235—Agent for treatment of immunodeficiency states]. However, the known pharmacologically active substance is incapable of overcoming MDR and differs in the chemical formula from the proposed hexapeptide of the linear structure lysyl-histidyl-glycyl-lysyl-histidyl-glycine. Empirical formula C28H44O6N12. Molecular weight 644.7 D. Therefore, the consideration touches quite different chemical substances possessing various actions: one substance, cyclo-lysyl-histidyl-glycyl-lysyl-histidyl-glycine, influences the immune system, the other lysyl-histidyl-glycyl-lysyl-histidyl-glycine influences multiple drug resistance.

One of the most active MDR inhibitors taken by us as the nearest prototype is a well-known drug containing a peptide as an active principle—Cyclosporine A [Russian Drugs Register, Vol. 16, pp. 983-984, 2008]. The drug has found its clinical application as an immunodepressant in organ and tissue transplantation. To overcome MDR, this drug is used only with experimental purposes in cell cultures. Cyclosporine A is a hydrophobic cyclic peptide with the following structural formula:

The known peptide Cyclosporine A inhibits MDR predominantly by way of competitive inhibition of the ATP-dependent transport proteins belonging to the P-gp family. Cyclosporine A has been shown to render little effect, if at all, on multidrug resistance proteins (MRPs) [Legrand O., Simonin G., Perrot J. Y. Blood. 1998. V. 91. No 12. 4480-44881. Cyclosporine A in in vitro experiments inhibits the activity of transport proteins at concentrations of 0.5-2 μM. It inhibits MDR in animal experiments, increasing efficacy of action of doxorubicin on tumour growth in mice. However, its clinical application to overcome MDR in patients appears impossible since in effective doses the drug renders a powerful toxic effect.

### SUMMARY OF THE INVENTION

The proposed invention is aimed at creating a therapeutic agent ensuring increased efficacy of overcoming multiple drug resistance, enhanced specificity of action in relation to transport proteins of multiple drug resistance (MRPs), increased safety, reliable relief of toxic reactions, and creation of acceptable dosage forms for prevention and therapy in patients presenting with manifestations of multiple drug resistance.

The mission assigned is herein solved by the fact that as a therapeutic agent used to overcome MDR to various chemical substances is a hydrophobic hexapeptide with the following structural formula: lysyl-histidyl-glycyl-lysyl-glycine. Empirical formula: C28II46O7N12.

### BRIEF DESCRIPTION OF THE DRAWINGS

FIG. 1. shows accumulation of calcein in the R388VR cells in the control (1) and after adding 12 nM peptide (2) in the presence on Co2+ ions in the medium (extracellular calcein quenching).

FIG. 2. shows calcein efflux from the preliminarily calcein-loaded R388VR cells in the control (1) and after adding 120 nM peptide (2).

FIG. 3. shows kinetics of R123 efflux from Hep-2 cells in the control (1), after adding Cyclosporine A, 1 μg/ml (2) and digitonin, 0.02% (3).

FIG. 4. shows kinetics of rhodamine R123 efflux from Hep-2 cells in the control (1) and after adding Cyclosporine A, 1 μg/ml.

FIG. 5. shows dependence of inhibiting the rate of R123 (R) expression from Hep-2 cells by hexapeptide and Cyclosporine A (Cs A) on their concentrations.

FIG. 6. shows survival of Hep-2 cells following exposure to 50 μM arsenate with 1 (2) and 100 (3) mg/ml hexapeptide.

FIG. 7. shows effect of hexapeptide on growth of Hep-2 tumour cells.

### DETAILED DESCRIPTION OF THE PREFERRED EMBODIMENTS

The proposed therapeutic agent contains as an active principle a synthetic hexapeptide with a molecular weight of 662.7 D.

The active principle is an odourless, white powder, readily soluble in water and isotonic sodium chloride solution. The substance is not alcohol- or chloroform-soluble.

The proposed therapeutic agent intended to overcome MDR contains the hexapeptide in a dose of 1-0.001% (10-0.01 mg). The manufactured dosage forms of the proposed agent contain a sterile hexapeptide solution for subcutaneous or intramuscular injections in ampoules. As a stabiliser, the agent contains a 1.0-0.5% solution (10-0.5 mg) of aminoacetic acid. A dosage form for subcutaneous or intramuscular injections is more preferable for the intended use as a 0.005% hexapeptide sterile solution in 1.0-ml vials and 0.5 aminoacetic acid as a stabiliser. Shelf-life is 2 years if stored at 4-8° C.

A spray dosage form for dosed intranasal use is more preferable in 10.0-20.0-ml bottles each containing 10-20 doses of the proposed agent. Each dose contains 0.05-0.1 mg of the hexapeptide and 5-10 mg of aminoacetic acid as a stabiliser. The solution for dosed intranasal administration contains benzalkonium chloride from 0.0040 to 0.00012 g/ml used as a preservative. The most preferable dosage form contains a single dose of 100 mg hexapeptide, 0.5% stabiliser aminoacetic acid and benzalkonium chloride 0.00010 g/ml. Shelf-life is 2 years if stored at 4-8° C.

The suppository dosage form intended for rectal or vaginal administration contains the active principle—hexapeptide at a single dose of 5-0.05 mg, aminoacetic acid as a stabiliser 20-0.5 mg (Russian Pharmacopoeial Monograph 42-599-92, ND 42-11253-00) or another Russia-registered similar-quality stabiliser, Tween 80 (Polysorbate 80) (PhM 42-2540-88) 120-150 mg, water for injections (PhM 42-2620-97) and hard fat (PhM 42-346-97, ND 42-8991-98) to obtain a suppository weighing 1.2-2.5 g. Shelf-life is 2 years if stored at 4-8° C.

The therapeutic agent has been devised in the Limited Liability Company Research and Production Enterprise “Bionox”, and from its composition, it does not to seem possible to immediately envisage such novel beneficial and patient-friendly properties thereof as listed below:

overcoming multiple drug resistance of cells;

specificity of action in relation to transport proteins of multidrug resistance (MRPs);

increased safety;

reliable relief of systemic toxic reactions;

creation of acceptable dosage forms for prevention and therapy of patients presenting with manifestations of multiple drug resistance.

The findings of preclinical and clinical studies of the proposed agent have shown that it neither induces a pronounced toxic reaction nor leads to development of side effects.

Experimental and clinical evidence showed that:

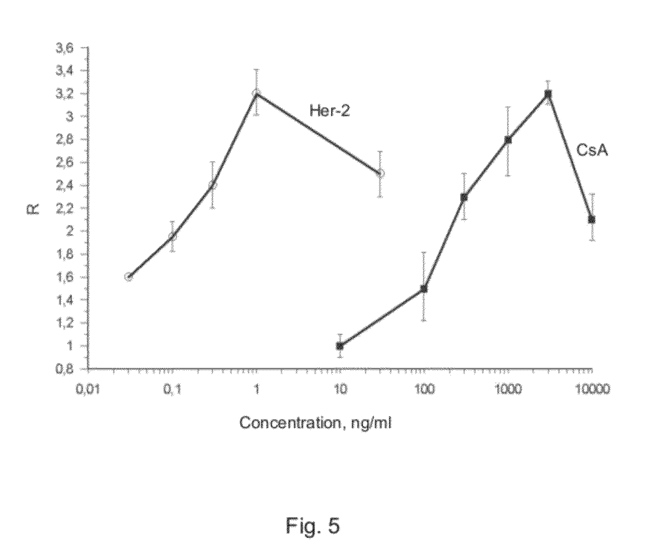

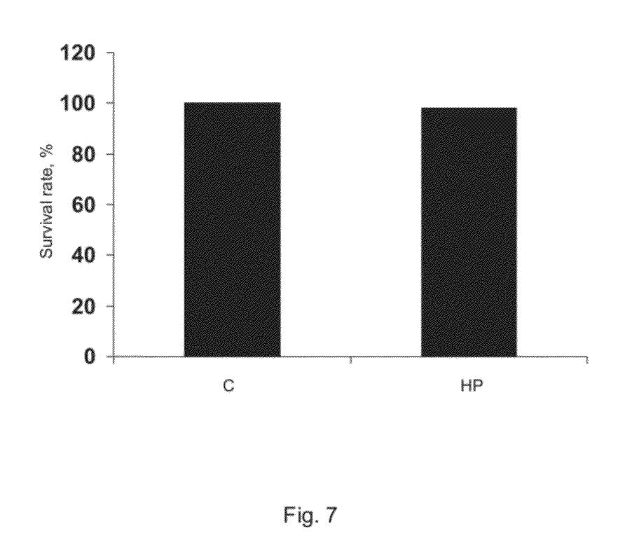

1. The hexapeptide ensures overcoming multiple drug resistance 3,000 times more effectively than the prototype substance (Example 3, FIG. 5) and possesses sufficient safety in relation to tumour growth (Example 4, FIG. 7).

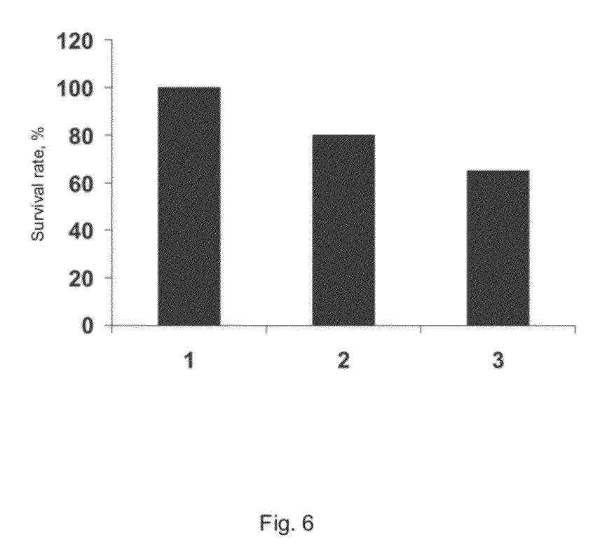

2. The proposed agent has specificity of action in relation to transport proteins of multidrug resistance (MRP). The prototype substance, i. e. Cyclosporine A, does not render a similar action. The hexapeptide at a concentration of 1.2 nM induces a 3-fold increase in sensitivity of human laryngeal cancer cells Hep-2 to the specific inhibitor of the protein mediating multiple drug resistance to cytostatic substance—arsenate (FIG. 6).

3. The hexapeptide possess exceptionally low toxicity and provides a wide reserve of therapeutic safety. A single dose 1,000-fold times exceeding the average therapeutic one (1.5 mg/kg) caused no death of laboratory animals.

4. The chronic toxicity test of the proposed agent reliably revealed its safety. Fluctuations of the haematological and biochemical parameters when the agent was administered to animals for one month daily at the therapeutic dose (1.5 μg/kg) or at a dose 10 times exceeding the therapeutic one (15 μg/kg) following discontinuation of the agent were found to have returned to the baseline levels.

5. When administered, the therapeutic agent produces no local irritating effect, possessing neither allergic nor mutagenic activity.

The therapeutic agent yielded good clinical results in comprehensive chemo-and radiotherapy of human cervical carcinoma, oesophageal cancer, and other malignant tumours. The proposed agent is to be administered in courses prior to and during chemoradiotherapy.

The efficacy of treatment is assessed by clinical and laboratory indices including the degree of the tumour's pathomorphism, development of toxic and radiation-exposure reactions, continuity of chemotherapy, the state of the oxidative-antioxidative and immune systems of the body. The drug should preferably be administered in courses at a single dose of 1-1.5 μg/kg body weight daily prior to commencing chemoradiotherapy for 5-10 days and concomitantly during courses of chemoradiotherapy. If it is necessary to relieve toxic reactions, the drug should be administered upon completion of chemoradiotherapy for 2-3 weeks.

Below are specific examples of the production of the active principle, description of biological and therapeutic properties of the proposed substance.

### Example I

Production of the Active Principle

The hexapeptide is synthesised according to the solid-phase synthesis on the automated synthesiser “Beckman-990”.

Aminomethyl polymerase (1.8 g, 1 mmol) is allowed to swell in chloroform for 30 minutes, followed by adding tert-butoxycarbonyl-Gly-OCH2C6H4CH2COOH (0.65 g, 2 mmol) with the help of N,N-dicyclohexylcarbodiimide (DCHC) (0.4 g, 2 μmol in 15 ml of chloroform for 2 hours). After washing with dimethylformamide, the polymer is treated with 20 ml of a mixture containing diisopropylethylamine:acetic anhydride 2:1 for 30 minutes. After washing with dimethylformamide and chloroform, the polymer is treated with 30 ml of a mixture consisting of trifluoroacetic acid:chloroform 1:1 for 20 minutes, washed with chloroform and neutralised with a 7% solution of diisopropylethylamine in dimethylformamide for 10 minutes, then the aminoacylpolymer is washed with dimethylformamide. The addition of tert-butoxycarbonyl-amino acid (BOC-amino acid) is carried out by means of symmetric anhydride of BOC-amino- acid: 4 mmol BOC-amino acid is dissolved in 5 ml of chloroform, allowed to cool to 0° C., followed by adding a 2-mmol solution of DCHC to 5 ml of chloroform. Following agitation for 10 minutes at 0° C., the mixture is added to the peptidylpolymer, pouring in 10 ml of dimethylformamide and stirring with the peptidylpolymer for 30 minutes at 28° C.

After the addition of the next amino acid, the peptidylpolymer is washed with dimethylformamide, chloroform, followed by removal of the protecting BOC-group.

Removal of the dinitrofenyl group: 2 g of the peptidylpolymer is agitated for 1.5 hours at ambient temperature in 40 ml of a 20% thiophenol solution in dimethylformamide. Then the peptidylpolymer is washed with chloroform and vacuum-dried.

Removal of the peptide from resin: 2 g of peptidylpolymer is placed into the reaction vessel of the device for dealing with anhydrous hydrogen fluoride, adding 1 g of n-cresol, cooled with liquid nitrogen, and vacuuming of the vessel is followed by adding 10 ml of anhydrous hydrogen fluoride, adjusting the temperature of the reaction vessel to −20° C. and stirring the polymer for 30 minutes, maintaining the temperature of the mixture consisting of CCl4—solid CO2, followed by placing the reaction vessel into a bath of iced water, then allowed to stand there while stirring for 30 minutes more. After this, anhydrous hydrogen fluoride is evaporated on the water-jet pump, the polymer is washed on the filter with diethyl ester. The peptide is then extracted with 20% acetic acid and freeze-dried.

Removal of the trifluoroacetyl group. The resin-removed peptide is processes with 60 ml of a 1 M aqueous solution of piperidine at 0 C for 2 hours. The reaction mixture is then lyophilised and desalinated by gel-filtration on the column Sephadex G-25 in 5% acetic acid.

### Example 2

Specificity of Action of the Proposed Substance as Compared with the Prototype.

Studying the effect of the hexapeptide (proposed substance) and Cyclosporine A (prototype substance) on specificity of action in relation to the transport proteins responsible for MDR.

The human laryngeal cancer cells Hep-2 in Dulbecco's modified Eagle's medium (hereinafter referred to as DMEM) (Sigma) supplemented with 10% foetal calf to serum (Sigma) and 40 μg/ml of gentamycin (Sigma) were sown in a 96-well culture plate, 0.1 ml per well, at a concentration of 50,000/ml, followed by adding the agents being examined and incubating for 72 hour at 37° C. in an atmosphere of 5% CO2 in air. After incubation, the cells were stained with 0.02% crystal violet solution (Sigma) in 20% ethanol for 10 minutes followed by discarding the stainer solution, washing the cells with water and adding a 0.1% SDS solution for the extraction of the stainer from the cells. The optical density was measured at 570 nm on the spectrophotometer Multiscan Plus. The effect was determined by the formula: No/Nk=(Do−D)/(Dk−D) 100%, where Do, Dk, D are optical densities in the experiment, control, and the background one, respectively, and No/Nk is the cell survival rate: the number of the live cells in the experiment in relation to the control.

R388 cells were grown in the abdominal cavity of the DBA2 mice. Vincristine-resistant R388 cells were obtained by growing them in DBA2 male mice exposed to 1 μg/g vincristine (Gedeon Richter, Hungary). Vincristine was administered intraperitoneally 24 hours after tumour inoculation (108 cells per mouse). After proliferation, the cells were transinoculated immediately followed by again administering 1 μg/g vincristine. The procedure was repeated six times. The thus obtained strain of cells was designated R388VR (vincristine-resistant).

All measurements of fluorescence were carried out on the spectrophotometer Perkin-Elmer MF-44 with continuous agitation at a temperature of 37° C. The wavelengths of excitation and emission for calcein were 493 and 515 nm, respectively. The Hep-2 cells (1-1.5 mln) were placed into a dish, preliminarily adhered to a transparent plastic plate, whereas the R388 cells (500 thou) were placed suspended. When determining calcein formation, calcein AM was added to the dish, registering the rate of calcein formation in the control sample, then adding to the dish the agents being examined, continuing to register increased fluorescence. The effect was assessed by the rate increment ratio following the addition of the agent. When studying the rate of calcein efflux from the cells, the latter were preliminarily loaded with the same fluorochrome in the presence of 1 μg/ml Cyclosporine A to inhibit their pumping out of the cells, then washed and stored until measuring on ice. When determining the calcein efflux from the suspended cells (R388VR), 2 μM Co2+ was added to the dish in order to quench fluorescence of extracellular calcein and thus registering calcein within the cells only. First, we determined the fluorochrome efflux rate in the control, then we added the agent being examined to the dish, continuing to register a decrease in calcein in the cells. The effect of exposure was assessed by the ratio of the efflux rates in the control and after adding the agent. All effects of the peptide were compared with those of Cyclosporine A.

The rate of calcein formation in the dish with calcein AM added without Co2+ ions makes it possible to determine the activity of the transport proteins expelling calcein AM out of cells, rather than that of the proteins capable of transporting calcein from the cells (MRP), since in this case total calcein is registered: both intra- and extracellular.

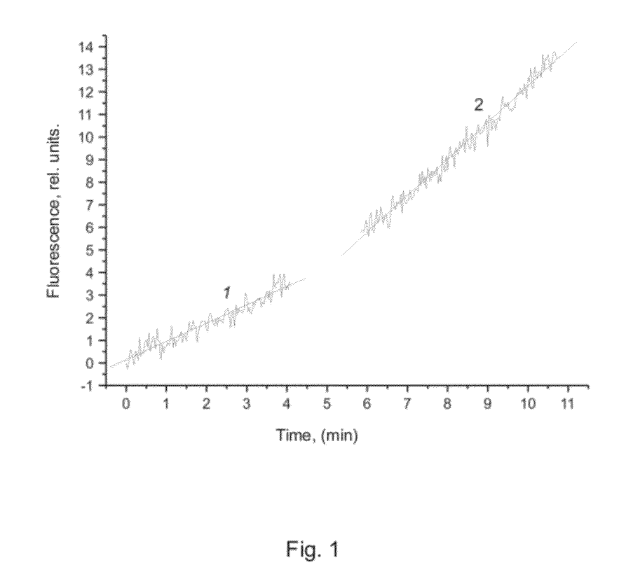

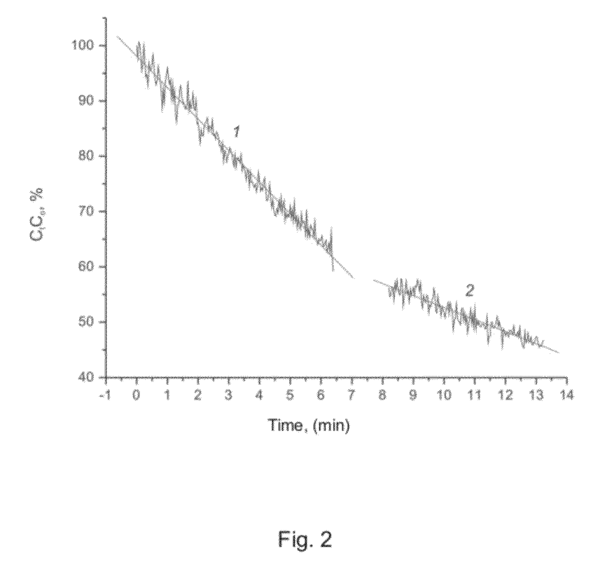

Cyclosporine A, being a predominant inhibitor of P-gp, effectively increases the calcein formation rate in the dish containing the R388VR cells wherein MDR is predetermined primarily by superexpression of P-gp. The calcein formation increment rate in the R388VR cells with Cyclosporine A amounted to 7±0.4 at its concentration of 0.8 μM and more. The hexapeptide does not act upon the calcein formation rate in the dish with the R388VR cells up to a concentration of 1.2 μM. However, in the presence of Co (extracellular calcein quenching) the hexapeptide at a concentration of 12 μM appears to double the calcein formation rate in these cells (FIG. 1). The results showed that the hexapeptide rendered no effect on activity of P-gp (pumping out of calcein AM in the R388VR cells), however inhibiting MRP which transports calcein from the cells. In fact, studying the calcein excretion rate from the preliminarily calcein-loaded loaded R388VR cells showed that the hexapeptide appeared to effectively inhibit MRP: the calcein excretion rate decreased 1.5-fold at the peptide's concentration being as low as 1.2 nM, with the rate inhibition ratio amounting to 2.6 at a concentration of 120 nM (FIG. 2).

On the contrary, Cyclosporine A exhibited extremely low efficacy in suppressing MRP, with the ratio equaling 1.3±0.1 at a concentration of 0.8 μM.

The obtained data demonstrate that the hexapeptide (proposed substance) does not act upon activity of P-gp (pumping out of calcein AM in R388VR cells), however inhibiting the MRP which in small amounts is also present in the R388VR cells and to transports calcein from the cell. Similar conclusions follow from the findings obtained on another model—Hep-2 cells. The multidrug resistance proteins in the Hep-2 cells, unlike the R388VR cells, contribute heavily to the development of MDR. Therefore, the hexapeptide in the Hep-2 cell inhibits MDR much more effectively than in the R388VR cells. Thus, the hexapeptide at as low a concentration as 1.2 nM increases twofold the rate of calcein formation in cells (with the simultaneously present Co2+ ions) and when the concentration of the peptide increases to 120 mM, the calcein formation rate increment ratio amounts to 2.6 (FIG. 2). Hence, the hexapeptide (proposed substance), unlike Cyclosporine A, renders a pronounced specific effect in relation to inhibiting the transport proteins of multiple drug resistance (MRPs).

### Example 3

Studying the Effect of the Hexapeptide and the Prototype Substance (Cyclosporine A) on Overcoming Multiple Drug Resistance of Tumour Cells Hep-2.

Activity of the multidrug resistance-associated transport proteins in cells was determined by the rate of rhodamine 123 (Rh123) (“ICN”, USA) expulsion out of cells. The ratio of the Rh123 efflux rates in health and in complete inhibition of the active transport minus one, i. e. (R−1)max, is known to equal the ratio of the active and passive transport and, consequently, characterises the activity of cell-contained proteins responsible for multiple drug resistance. Hep-2 cells were sown in the amount of 1-1.5 min in Dulbecco's modified Eagle's medium (DMEM)+10% foetal calf serum with gentamycin on the plates (50×9×2 mm in size) placed in dishes 6 cm in diameter, with the medium volume amounting to 8 ml, immediately followed by incubation in a CO2 incubator. After 24-hour incubation in CO2 at 37° C., the cells were washed in RPMI 1640 medium and loaded with Rh123, 0.5 μg/ml in the presence of the inhibitor of transport proteins Cyclosporine A, 3 μg/ml in RPMI 1640 medium supplemented with 5% foetal calf serum for 60 min at 37° C. After incubation, the cells were subsequently washed three times (each for 10 min) from the stainer with cold (2° C.) saline supplemented with 1% foetal calf serum. After washing and till the moment of measuring, the cell-containing plates were stored in Hanks solution with 0.5% foetal calf serum, put on ice. The washed plate with cells was placed in a cuvette of the spectrophotometer MF44 Perkin-Elmer with Hanks solution and 0.5% foetal calf to serum, volume 3 ml, at 37° C. and used to determine an increase in the amount of Rh123 in the medium with continuous shaking. The wavelengths of excitation and emission amounted to 488 and 520 nm, respectively. At the end of the experiment in order to determine the maximum amount of Rh123 in the cells, the latter were destroyed by adding to the cuvette 0.02% digitonin (“Sigma”, USA).

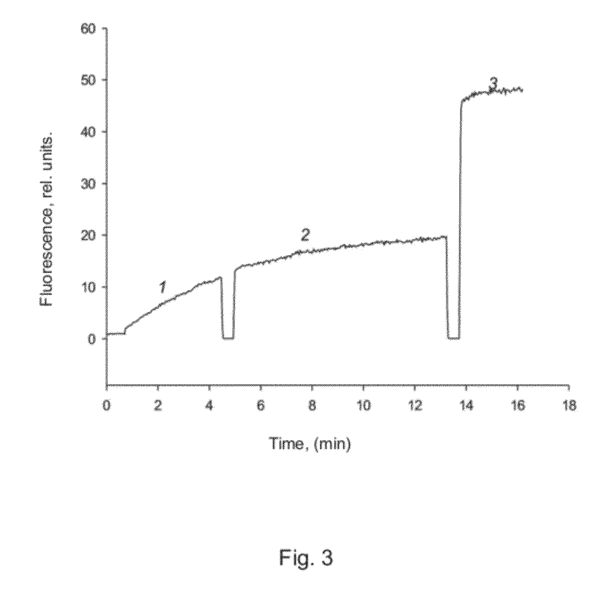

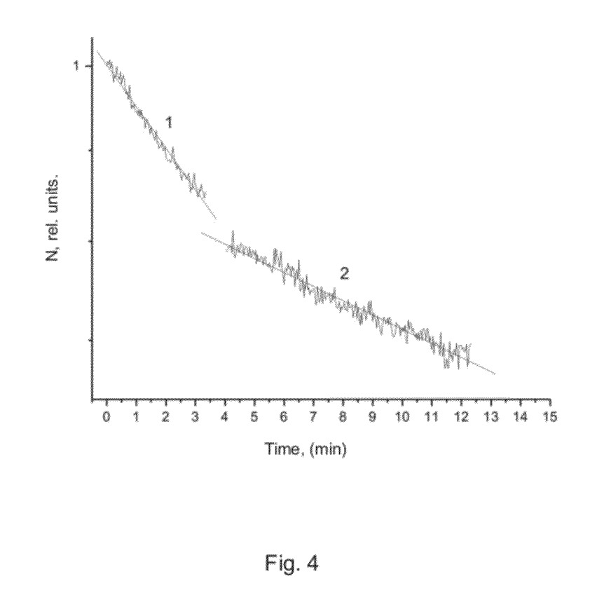

While studying the effect of the inhibitors on the release of Rh123, to the cuvette after several minutes of the normal excretion of the stainer we added the inhibitor in the required concentration. Determining the control and inhibited constants of rhodamine excretion rates on one plate decreases the error of determining their ratio, necessary for obtaining the R value. FIG. 3 shows the kinetics of Rh123 increase in the control medium (1), after adding Cyclosporine A (2) and digitonin (3). The total amount of rhodamine in the cells was determined by the difference of the fluorescence levels after adding digitonin and before adding cells to the cuvette. The relative amount of Rh123 in the cells (N) was determined according to the following formula: N=1{It−I0/Imax−I0}, where It, I0, Imax are the values of fluorescence of Rh123 in the medium at the time moments t, 0 and the peak fluorescence after adding digitonin, respectively. As can be seen from FIG. 4, the amount of Rh123 in the cells in the control and after adding the inhibitor decreases exponentially, since on a semilogarithmic scale these kinetics are described by straight lines. In the given example Cyclosporine A at a dose of 1 μg/ml decreased 2.8-fold the rhodamine 123 excretion rate constant: R=2.8.

The above described method was used to determine the effect of the proposed hexapeptide as compared with that of the known MDR inhibitor Cyclosporine A on the efflux of Rh123 from the Hep-2 cell. The effect of each concentration of the agents on Rh123 excretion was determined in several experiments. FIG. 5 shows the mean values of R, with standard deviations, for the concentrations where there were more than 3 replications.

As can bee seen from FIG. 5, the hexapeptide (HP) maximally inhibits Rh123 excretion at an optimal concentration of 1 ng/ml. The R value for the hexapeptide equals 3.2. A similar value of R for Cyclosporine A is reached at a concentration more than three thousand times greater than that of the proposed hexapeptide.

Hence, the proposed substance—hexapeptide ensures overcoming multiple drug to resistance more than three thousand times more effectively as compared with the prototype substance, i. e., Cyclosporine A.

### Example 4

Oncological Safety and Harmlessness of the Proposed Hexapeptide

Human laryngeal cancer cells Hep-2 in DMEM medium supplemented with 10% foetal calf serum and 40 μg/ml gentamycin were inoculated in 2-ml penicillin-containing vials, 200 thousand per each vial. The hexapeptide at a dose of 1 μg/ml was added 3 hours after cell inoculation (when they had already adhered to glass) and left in the medium for the whole period of incubation. Three days after inoculation, the cells were detached from glass with trypsin, followed by calculating the number of the living cells: unstained with a 0.04% solution of trypan blue.

Hence, the hexapeptide does not bring about stimulation of tumour cell growth. On the contrary, it increases sensitivity of the tumour cells to the cytostatic preparation arsenate and inhibits survival of the Hep-2 cells (FIG. 6). FIG. 7 shows evidence regarding the effect of the hexapeptide on the Hep-2 cells growth.

It is seen that the hexapeptide at a concentration of 1 μg/ml virtually does not influence the growth of the Hep-2 tumour cells (FIG. 7).

Thus, the obtained findings demonstrate that the hexapeptide (proposed substance) enhances the effect of the cytostatic agent and is safe in relation to stimulation of tumour growth.

### Example 5

Female patient R., 37 years old. Clinical diagnosis: stage III cervical carcinoma (T3NX-1M0). Examination of the patient included clinical and laboratory assessment both at admission and during treatment. Her general condition was of moderate severity. On the background of the virtually normal parameters of the oxidative-antioxidative system, the patient was found to have acute immunodepression as judged by the parameter of the percent content of the main subpopulations of T-lymphocytes. The percentage of CD3, CD4, CD8, CD16-positive cells was decreased relative to the average statistical normal values 2-fold. The woman prior to antineoplastic treatment had received a course of 5-time administration of the hexapeptide in the form of 1 ml of 0.005% solution subcutaneously daily for 5 days. Chemoradiotherapy was carried out according to the regimen: 500 mg of 5-fluorouracil (total dose 2,500 mg). During the whole course of chemoradiotherapy, the woman received 1 ml of 0.005% solution of the hexapeptide (biopoetin) 5 times daily. Two days after discontinuation of 5-fluorouracil, the patient underwent remote irradiation at a single dose of 4 Gy on the background of intravenous administration of 30 mg of cysplastin for 3 days (total dose of cysplastin amounting to 90 mg). Then radiotherapy was continued in the mode of a daily multifractionated dose. Irradiation was carried out up to a focal dose of 20 Gy onto the area of the primary focus and zones of regional metastatic spreading, followed by additional intracavitary gamma-therapy (10 fractions 5 Gy each) and remote irradiation was continued onto the zones of parametrial metastatic spreading up to the total dose amounting to 44 Gy. No pronounced side reactions were observed, thus making it possible to maintain discontinuity of the courses of chemoradiotherapy. No events of MDR development were observed. At the same time, the average statistical indices of chemoradiotherapy of patients suffering from stage III cervical carcinoma demonstrate the development of specific reactions requiring a long-term break of therapy in 33-34%, the development of epithelitis in 78-87%, islet epithelitis in 55-58%, and in 15-20% scarious epithelitis requiring 4-5-month treatment. It is important that the indices of immunity and the total blood count after chemoradiotherapy in the woman did not virtually alter, whereas in the control group not receiving the hexapeptide solution a further decrease in the above indices was observed. The treatment performed resulted in complete resorption of the tumour.

Hence, the proposed agent appears to reduce manifestations of toxicosis, the development of side effects and radiation reactions to chemoradiotherapy.

### Example 6

Patient N., 52 Years.

Clinical diagnosis: stage III oesophageal cancer (T3-4N0-2MOP3). At admission, the patient's general condition of moderate severity. The laboratory indices of the total blood count without pronounced alterations. Noted was a considerable disbalance of the indices of the oxidative-antioxidative system on the background moderate-degree immunosuppression. The state of the oxidative-antioxidative system in to the patients was assessed by intensity of lipid peroxidation via measuring blood serum concentration of malonic dialdehyde (MDA, μmol/L) and endogenous antioxidants: lactoferrin (LF, mg/L), caeruloplasmin (CP, conventional units), and the antioxidant enzyme catalase (CAT, conventional units). The analysis of percentage content of the main subpopulations of T-lymphocytes was carried out to superficial receptors of CD3 (T-lymphocytes), CD4 (T-helpers/inducers), CD8 (T-suppressors/cytotoxic lymphocytes), CD16 (natural killers), CD25 (activated lymphocytes expressing the receptor to interleukin-2), CD72 (B-lymphocytes). At admission to the Chemotherapy Department, the patient was found to have substantial disbalance of the oxidative-antioxidative system: MDA-3.5 (norm 3.0±0.1), CAT-450 (norm 576±48), CP-0.59 (norm 0.52±0.04), LF-3.8 (norm 1.0±0.4). At the same time, the indices of the lymphocytic link were virtually within the normal values. Since CP and LF are proteins of the acute phase of inflammation, an increase in the blood serum reflected the presence of an inflammatory process accompanying the development of oesophageal cancer. Prior to chemoradiotherapy, the patient received intramuscular administration of the hexapeptide daily at a single dose of 1 ml of 0.1% solution 5 times a day for 5 days. Comprehensive chemoradiotherapy was carried out according to the following regimen: 750 mg of 5-fluorouracil (5-FU) was administered intravenously for 5 days (total dose 3,750 mg), two days after the end of administration of 5-FU, the patients was irradiated according to the regimen of dynamic fractionation of the dose at a single dose of 4 Gy on the background of intravenous administration of cysplastin at a dose of 30 mg for 3 days (total dose of cysplastin 90 mg, total focal dose 12 Gy). During the whole period of chemoradiotherapy, the patients was given hexapeptide at a single dose of 100 μg twice daily (in the morning and evening). Commencing from day 11, radiotherapy was continued in the mode of the classical fractionated dose, with the single dose equaling 2 Gy (total focal dose 40 Gy). No pronounced side or post-irradiation reactions were observed which provided continuity of the courses of chemoradiotherapy. During the whole course of chemoradiotherapy, the indices of the natural cellular and T-lymphocytic link of immunity, production of acute inflammation phase proteins did not alter substantially. The patient showed improved indices of the oxidative-antioxidative system and a decrease in the malonic dialdehyde concentration to 3.2 μmol/L. No symptoms of MDR were observed. The carried out treatment in the patient presenting with stage III oesophageal cancer resulted in considerable improvement of the general condition, reduction of toxicosis events, removal of side effects and radiation reactions to chemoradiotherapy, and eventually complete resorption of the tumour.

Hence, the agent proposed reduces toxicosis events, the development of side effects and reactions to chemoradiotherapy. The treatment performed resulted in complete tumour resorption.

12-month follow-up of the patient having undergone the course of treatment with the use of the proposed agent revealed no disease relapses.

### Example 7

Female Patient P., 54 years old.

The disease was manifested by retrosternal pain, weakness, weight loss. Examination based on endoscopy, roentgen-contrast study revealed cancer of the median and lower sternal oesophageal portions approximately 10 cm in length with metastatic involvement of regional lymph nodes. Histology of the tumour biopsy sample revealed squamous-cell cancer. Prior to chemoradiotherapy, the patient was given the hexapeptide in the form of a suppository at a dose of 5 mg five times daily for 5 days.

The first stage of combined-modality treatment consisted of two courses of polychemotherapy.

During the whole period of chemoradiotherapy, the patient received treatment with the proposed agent in the form of rectal suppositories 5 μg twice daily (in the morning and evening).

No markedly pronounced manifestations of toxicosis were observed during polychemotherapy, with only moderate neutropenia accompanied by a decrease in the T-cell immunity indices, sporadic diarrhea, nausea, and dizziness.

The check-up examinations revealed no oesophageal tumour perceived, with substantially diminished size of the regional lymph nodes. Analysing the histological and biopsy material failed to confirm the presence of tumour cells. The histological to study of the biological material of subtotal oesophageal resection made it possible to reveal the whole curative pathomorphism of the tumour. No metastases into lymph nodes were detected.

### Example 8

Female patient P., born in 1939, was admitted with the diagnosis: breast cancer T4cN2M0 with cutaneous ulceration of the mammary gland.

From anamnesis: the patient had fallen ill about 2 years ago when she for the first time had noticed a tumorous formation in her left breast, which gradually increased in size.

On admission: complaints for the presence of a tumour in the left breast with a bleeding ulceration, general weakness, headache, nausea, pain in the left breast.

Findings of examination:

The left mammary gland visually showed on the border of the upper quadrants a knobby tumorous formation in the form of a carbuncle with ulceration in the centre, accompanied by bloody discharge and bad smell. The mammograms of the left breast showed a tumorous node more than 10 cm in diameter with skin infiltration and destruction in the centre (ulcer).

Mammoscintigraphy: foci of hyperfixation of 99Tc, measuring about 10 cm in diameter in the left breast and about 6 cm in diameter in the left axillary region. The findings of ultrasonographic study revealed a round globular formation with hypoechogenic contours 92 mm in diameter on the border of the upper quadrants of the left breast. Histologically: tumour cells of a poorly differentiated adenocarcinoma. Conclusion: breast cancer with destruction of the tumour, a metastasis to axillary lymph nodes on the left.

Onto the area of the left breast from two tangential opposed fields and with the figured field onto the zones of regional lymph outflow on the left radiotherapy was begun using linear accelerator (LU-20-SL with the deceleration-radiation energy of 6 MeV) in the mode of average dose fractionating, at 3 Gy daily 5 times a week onto the area of the tumour up to the total focal dose (TFD) 45 Gy equivalent to the mode of the conventional fractionation consisting of 2 Gy a day, with TFD 60 Gy, and onto the zones of regional lymph outflow on the left (supraclavicular, subclavicular, axillary and parasternal sites) at a single dose of 3 Gy up to the TFD 33-36 Gy equivalent to 44-48 Gy of the conventional fractionation.

The patient's state after the irradiation course was satisfactory, with the tumour ulceration being at the scarring stage and virtually no discharge. Two weeks later, polychemotherapy was begun according to the CMF regimen carried out for 4 weeks once a week. During 5 days prior to commencing chemotherapy, the woman daily received the hexapeptide in the form of an intranasal dosed spray 5 times a week at a single dose of 100 μg. The CMF regimen included intravenous drip administration of 1 g of 5-fluorouracil and 40 mg of methotrexate and intramuscular administration of 1 g cyclophosphane. During polychemotherapy, the patients daily received the hexapeptide in the form of an intranasal dosed spray, with the single dose equaling 100 μg. The patient tolerated chemoradiotherapy satisfactorily: WBCs—4.5×109, blood platelets—200×103/L. The patient was discharged from hospital in a satisfactory condition.

A repeat course of CMF. The patient received intravenously 40 mg of methotrexate and 1 g of 5-fluorouracil and intramuscularly 1 g of cyclophosphane. The state after polychemotherapy was satisfactory. A cicatrix formed on the site of the previous breast ulceration. Regional lymph nodes were not perceived, the breast was soft to the touch, with no fresh infiltrates. On the mammograms, the tumour node as compared with the findings of mammography obtained at the beginning of treatment had decreased to 5 mm in diameter. Then sequentially a course of polychemotherapy was carried out according to the same regimen and in the same doses. During 5 days prior to commencing chemotherapy, the patient daily received hexapeptide in the form of an intranasal dosed spray 5 times a day at a single does of 100 μg. During polychemotherapy, the patient daily received hexapeptide in the form of an intranasal dosed spray 5 times a day at a single does of 100 μg.

The patient was discharged home to be followed-up by the district oncologist, and than hospitalised again to undergo a fourth course of polychemotherapy. Her condition was satisfactory, with a smooth scar on the ulcer site, no infiltrative events, the axillary and subclavian lymph nodes not perceived, and the right mammary gland unaltered. The mammograms showed that the tumour node in the form of fibrous tissue had decreased to 3.8 cm as compared with the last mammographic findings. The clinical blood count with nothing to be particularly noted.

A 2-week chemotherapeutic course was begun according to the CMF regimen. During 5 days prior to commencing chemotherapy, the patient received daily the hexapeptide in the form of an intranasal dosed spray 5 times a day at a single does of 100 μg. During polychemotherapy, the patient daily received the hexapeptide in the form of an intranasal dosed spray 5 times a day, with the single dose equaling 100 μg.

The patient tolerated chemoradiotherapy satisfactorily: WBCs—4.5×109, blood platelets—200×103/L.

The clinical and roentgenological study (physical examination, mammography, ultrasonography of the mammary glands and abdominal cavity, roentgenography and tomography of the lungs and mediastinum, osteoscintigraphy, clinical and biochemical blood count and urinalysis) showed no findings suggesting either a breast tumour relapse or metastases.

The 5th 2-week course of polychemotherapy was performed according to the CMF regimen which was well tolerated by the patient. No development of MDR was noted. Within 5 days prior to commencing chemotherapy, the patients received daily the hexapeptide in the form of an intranasal dosed spray 5 times a day at a single does of 50 μg. During polychemotherapy, the patient daily received hexapeptide in the form of an intranasal dosed spray 5 times a day with the single dose amounting to 50 μg. The patient's condition was satisfactory. The clinical-and-roentgenological examination showed no evidence suggesting progressive tumour growth. Once again, a 2-week course of polychemotherapy was performed according to the CMF regimen also satisfactorily tolerated by the patient. She was then discharged home to be followed-up by the district oncologist. The check-up examination (15 months after the beginning of treatment) showed no signs of the disease's relapses.

1. A therapeutic agent intended to overcome multiple drug resistance, comprising a peptide and a target additive, patentably distinctive in that as a peptide it contains a hexapeptide with the following structural formula: lysyl-histidyl-glycyl-lysyl-histidyl-glycine.

2. The therapeutic agent according to claim 1, wherein it is manufactured in the form of a solution for subcutaneous and intramuscular injections and contains aminoacetic acid as a target additive.

3. The therapeutic agent according to claim 1, wherein it is made in the to form of a spray intended for dosed application and contains as target additives aminoacetic acid and benzalkonium chloride.

4. The therapeutic agent according to claim 1, wherein it is made in the form of a suppository containing as target additives aminoacetic acid, Tween 80 (Polysorbate 80), water for injections, and hard fat.
